(19) United States
(12) Patent Application Publication
(10) Pub. No.: US 2024/0036021 A1
(43) Pub. Date: Feb. 1, 2024

(54) SYSTEM AND METHOD FOR CONTROLLING CELL FUNCTIONING AND MOTILITY WITH THE AID OF A DIGITAL COMPUTER

(71) Applicant: XEROX CORPORATION, NORWALK, CT (US)

(72) Inventors: Krishnan Thyagarajan, Sunnyvale, CA (US); Christopher Somogyi, Wimberley, TX (US)

(21) Appl. No.: 17/876,065

(22) Filed: Jul. 28, 2022

(51) Int. Cl. G01N 33/00; C12N 5/04; A01H 4/00; C12N 13/00

(52) U.S. Cl. CPC G01N 33/0098; C12N 5/04; A01H 4/00; C12N 13/00; G01N 2015/1006

(57) ABSTRACT

A feedback-based system and method that identifies characteristics of one or more cells and utilizes the characteristics to initiate and adjust a field applied to the one or more cells to control the cells' functioning and motility is provided. Machine learning can be leveraged to automatically identify characteristics of the one or more cells and adjust the parameters of the field based on the characteristics. Sensors are utilized during the application of the field to monitor characteristics of the one or more cells and parameters of the field. Specifically, characteristics of at least some of the cells are measured at different points, and the measurements are used to determine whether the desired effect has been achieved or whether unintended consequences are taking place. Based on the measurements, parameters of the field can be adjusted to achieve the desired effect on cell functioning, cell motility, or both.

### FIELD

This application relates in general to biotechnology, and in particular, to a system and method for controlling cell functioning and motility with the aid of a digital computer.

### BACKGROUND

As human population increases, there is an added pressure on biotechnology to produce results necessary to satisfy the population's growing needs and achieve increased control over functioning of both single cellular and multicellular organisms. For example, agriculture is under increasing pressure to increase output in regards to four areas: crop yield, rate of crop yield, crop yield per unit area, and nutrient availability in the crops harvested. Achieving improvements in these four areas is complicated by the breadth of organisms that now fall under the definition of a crop. Whereas previously the definition of crop was limited to plants, there is a growing amount of research on agricultural utilization of other organisms, including algae, both to be used as food and for other purposes such as production of biofuels. This breadth of organisms whose cultivation needs to be improved puts significant pressure on availability of land suitable for land-based agriculture as well as other infrastructure used for other agricultural techniques, such as hydroponics, and to relieve such pressure, increased control over the cultivated organisms is necessary.

A similar pressure exists to increase control over organisms used in other areas of biotechnology. For example, tissue engineering involves using cells to generate biological tissues and holds enormous promise for generation of organs and other kinds of tissues for medical and other needs. To achieve the desired result, one needs to control not only the rate of and the kind of cellular growth and proliferation, but also the direction in which particular kinds of cells move, with desired tissue structure often requiring particular kinds of cells at a particular location. Accordingly, as the demand for the products of tissue engineering increases, so does the need for increased control over cell functioning and motility that would allow to achieve increased output for tissue engineering.

Still other areas of biotechnology are under similar pressures to increase control over functioning of cells, including those making up hyperaccumulators and similar organisms. Hyperaccumulators are organisms that are capable of growing in an environment with a concentration of metals that is toxic to other plant species, absorbing the metals and accumulating the metals in their tissues. Further, while the traditional definition of hyperaccumulators is limited to plants, other kinds of cells with similar properties exist. For example, vanadocytes are a type of blood cells found in ascidians that accumulate high levels of vanadium, which is both toxic to a large number of organisms and has important industrial applications. These properties are of a significant value in both phytoremediating the environment contaminated by the metals and in obtaining the metals for industrial purposes, and increased control of over the function of hyperaccumulators and similar organisms is necessary as demand for both of these outcomes grows.

Existing techniques for manipulating cell growth, motility, and other functions are not adequate for addressing these challenges. For example, current state of the art in agriculture uses fertilizers, green houses, and light of certain pre-determined wavelengths to increase plant yield in a fixed volume. Likewise, electromagnetic radiation has been applied to plants and other organisms to accelerate their growth, as described in Li, X., Luo, J., Han, K. et al. Stimulation of ambient energy generated electric field on crop plant growth. Nat Food 3, 133-142 (2022), available at https://doi.org/10.1038/s43016-021-00449-9. However, these such techniques often do not account for differences in the characteristics of the organisms that are being manipulated, which may not result in intended consequences. Further, absent constant manual supervision, which may not be able to observe all desired characteristics, these techniques do not allow to assess in near-real time their effect on the organism being manipulated and adjust the treatment of the organism if unintended effects appear.

Accordingly, there is a need for a way to manipulate function and motility of organisms, including single cellular and multicellular organisms, in a way that accounts for their unique characteristics and that adjusts the manipulation to the changes that are happening to the organism.

### SUMMARY

A feedback system that identifies characteristics of one or more cells and utilizes the characteristics to initiate and adjust a field applied to the one or more cells to control the cells' functioning and motility is provided. In one embodiment, the system leverages machine learning to automatically identify characteristics of the one or more cells and adjust the parameters of the field based on the characteristics. Sensors are utilized during the application of the field to monitor characteristics of the one or more cells and parameters of the field. Specifically, characteristics of at least some of the cells are measured at different time points and the measurements are used to determine whether the desired effect has been achieved or whether any unintended consequences are taking place. Based on the measurements, parameters of the field can be adjusted to achieve the desired effect on the function or motility of the cells (or both function and motility). The cells being manipulated can be part of a multicellular or unicellular organism, including both eukaryotic and prokaryotic cells, and can include terrestrial plants, aquatic plants, fungus, algae, and animals, though still other kinds of cells are possible. The cells can being manipulated can also be grown in vitro, being part of eukaryotic or prokaryotic cellular cultures, for purposes of creating biological material in areas such as tissue engineering. By providing the increased control over the functioning and motility of cells, the disclosed system and method help achieve increased output in many areas of biotechnology, including food production, biofuel production, phytoremediation and extraction of valuable substances from contaminated medium, and tissue engineering.

In one embodiment, a feedback-based method for controlling cell functioning and motility is provided. One or more sensors are used by a controller to determine characteristics of one or more of the cells within a receptacle at multiple time points. Parameters for one or more fields to be applied to one or more of the cells at the multiple time points via one or more field generators positioned with respect to the receptacle are determined by the controller based on the characteristics determined at those time points, each field generator including one or more of a magnet, transducer, electromagnet, or a pair of electrodes, the parameters including least one of amplitude, frequency, wavelength, phase, waveform, and duration. Application of the one or more fields by the one or more field generators is controlled by the controller based on the parameters, wherein each of the applied fields causes a change in at least one of functioning and motility of the cells to which that field is applied.

Still other embodiments of the present invention will become readily apparent to those skilled in the art from the following detailed description, wherein is described embodiments of the invention by way of illustrating the best mode contemplated for carrying out the invention. As will be realized, the invention is capable of other and different embodiments and its several details are capable of modifications in various obvious respects, all without departing from the spirit and the scope of the present invention. Accordingly, the drawings and detailed description are to be regarded as illustrative in nature and not as restrictive.

### BRIEF DESCRIPTION OF THE DRAWINGS

FIG. 1 is a block diagram showing a system for controlling cell functioning and motility with the aid of a digital computer in accordance with one embodiment.

FIG. 2 is a flow diagram showing a method for controlling cell functioning and motility with the aid of a digital computer in accordance with one embodiment.

FIG. 3 is a block diagram showing a top view of a device for feedback-based field application for use in the system and method for controlling cell functioning and motility in accordance with one embodiment.

### DETAILED DESCRIPTION

Control over cellular functioning and motility can be accomplished with by applying an electric, electromagnetic, or magnetic fields to the cells, monitoring the effects of the applied fields, and adjusting the application of the field as necessary for achieving a desired goal in a monitoring-based feedback loop. As described below, the cells that can be the subjected to the application of the field can be a part of a multicellular organism such as a plant, can be one or more unicellular organisms such as unicellular fungus, or can be single cells or group of cells (such as cell colonies) being manipulated in an in vitro environment for purposes such as tissue engineering or extraction of elements from the medium in which those cells are located, though still other types of cells are possible. Further, the application of the field as described below can affect only the functioning of the cells (such as cell growth, division, death, and differentiation), only the direction in which the cells move (motility), or both the functioning and the motility of the cells.

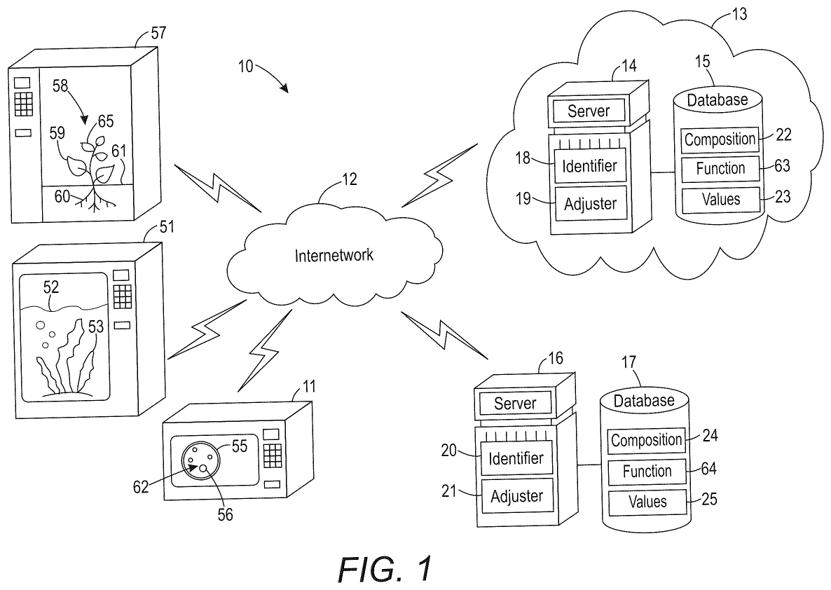

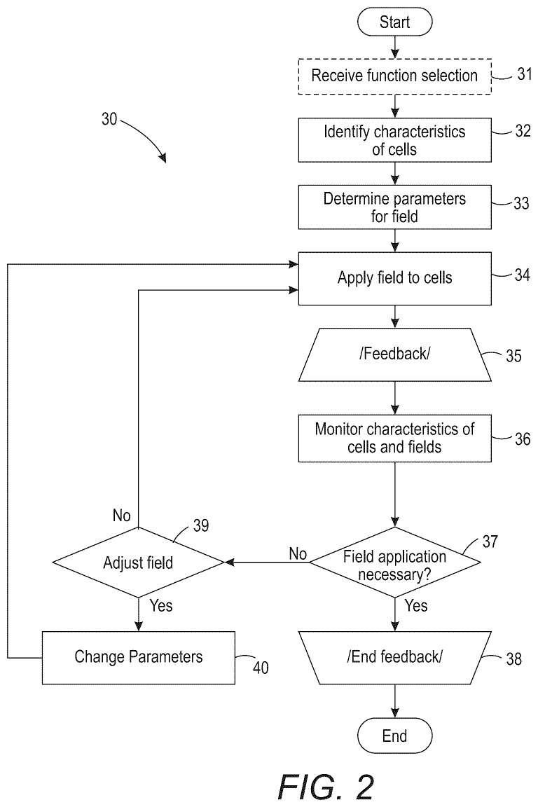

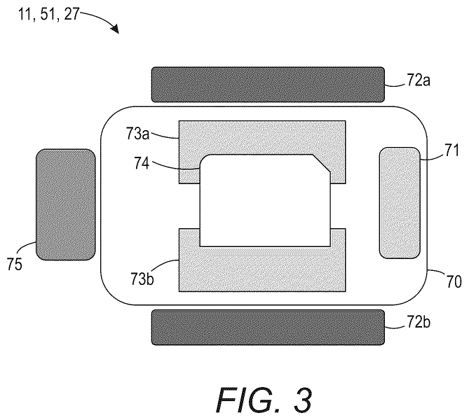

Utilizing a feedback system during application of the fields helps maintain the progression toward the desired result. FIG. 1 is a block diagram showing a system 10 for controlling cell functioning and motility with the aid of a digital computer in accordance with one embodiment. A field application device 11, 51, 57 can initiate and maintain a range of biological processes within both multicellular organisms and individual cells as well as cause motion of individual cells in a desired direction through application of a field, such as a magnetic, electric, and electromagnetic fields. The field is specifically tailored based on identity and characteristics of the cells to which the field is being applied, and is adjusted based on continued monitoring of the characteristics of the cells and the field using the sensors in the device 11, 51, 57 to control progression towards a desired result—this monitoring-based feedback loop is described in detail with reference to FIG. 2. The field application device 11, 51, 57 can be a standalone device or can be incorporated into an appliance, an incubator, or a structure such as a greenhouse and is described in detail below with respect to FIG. 3.

The field application device 11, 51, 57 can take many form suitable for different kinds of cells being manipulated. For example, the embodiment of the field application device shown with reference numeral 57 can be used to apply the field in the monitoring-based feedback loop to cells in a terrestrial environment, such as cells that are part of terrestrial plants 58. In particular, the device 57 can apply the fields towards leaves 59, roots 60, stem 65, or other part of the plants 58, which among other effects, can affect the rate of uptake of substances such as nutrients, water, or other substances of interest from the medium 61 in which the plant is located and distribution of such substances throughout the plant 58. The substances absorbed can either accelerate the growth of at least some parts of the plant 58, or if the substances are of intrinsic value (such as particular minerals or rare metals) be absorbed by the plant 58 and later harvested from the plant biomass. If intended, the rate of nutrient uptake can lead to overgrowth of certain part of the plant 58, leading to the plant's 53 death. Such a result can be desired if the plant 58 to which the field is being applied is a weed or another undesirable plant that is growing among plants 58 whose presence is desired and which can be left unharmed by not applying the field to them. Still other effects of field application are possible.

The medium 61 in which the plant 58 is planted can be either a solid medium, such as soil, or the liquid medium, if a hydroponics container is being used to grow the plants. Further, the device 57 can enclose the plant 58 in a variety of ways. In one embodiment, the device 57 can enclose the plant 58 that is plotted in the ground or in a hydroponics container on four sides, leaving the plant exposed to air and sunshine from above. In a further embodiment, the device 57 can enclose the plant 58 from less than four sides. In a still further embodiment, if the plant is in a controlled environment such as in a greenhouse, the medium 61 in which the plant 58 can also be within the device 57, and the device 57 can surround the plant from all sides. In this embodiment, the top of the device 57 could be transparent to allow natural light to reach the plant, or the device 58 could include artificial lighting, such as light-emitting diode (LED) or other kinds of light capable of emitting solar-spectrum light. Likewise, when surrounding the device from all sides, appropriate openings for ventilation would be included in the device 58 to allow for levels of oxygen and other gases necessary for growth of the plant 58 or achievement of other goals. While only a single plant 58 is shown inside the device 57 with reference to FIG. 1, in a further embodiment, multiple plants 58 can be manipulated by the same device 57 at the same time, with different field generators being directed at different plants. Further, while the plant 58 is shown as including both roots and a shoot, plants in a different stage of development, such as seeds (which can be made to germinate due to the application of the field), can be similarly manipulated using the field. Such control over plant growth and death can help increase the output in the four areas of agriculture described above, helping alleviate shortages of plants that are suitable for use as least one of food or for biofuel production. Further, other kinds of organisms, either multicellular or single-cellular organisms, such as can be manipulated in a similar way in the device 57.

A further embodiment of the field application device 51 can be used for applying the field in the monitoring-based feedback loop to cells in an aquatic environment, such as an aquatic plant 53. In this embodiment, the device 51 can contain the aqueous medium 52 in which the plant 53 is suspended, and the application of the field can increase the rate at which the plant 53 absorbs substances from the medium 52 and affect how the absorbed substances are distributed through the plant 53. The absorbed substances, if they are nutrients, can accelerate the growth of the plant 53. Alternatively, if the absorbed substances include rare metals or other valuable elements, the absorbed substances can be harvested from the biomass of the aquatic plants 51. Similarly to terrestrial plants 58, the applied field can be also used to kill the plant 58. Further, other kinds of organisms, either multicellular or single-cellular organisms can be manipulated in a similar way while in the aqueous medium 52 of the device 51. For example, cells such as vanadocytes can be suspended in the medium 52, be used to extract valuable substances such as vanadium from the medium 52 upon the application of the field by the device 52, with the substances being harvested from the cell biomass upon the extraction of the cells from the device 51.

A still further embodiment of the field application device 11 can be used to cause in cell functioning and motion of individual cells and groups 56 of cells for purposes such as tissue engineering. Thus, a scaffolding 55 (such as a petri dish) that includes cellular culture (such as cells or groups of cells in colonies 56) on a medium 62 can be placed within the device 11 and the application of the field (including a purely electric, purely magnetic, electromagnetic, AC/DC, or other kinds of field) in the monitoring-based feedback loop can affect the functioning of the cells, such as growth, absorption of substances from the medium 62, division, differentiation from a pluripotent cell (such as a stem) to a differentiated cell (such as a neuron), or death. The cells in the scaffolding 55 can be prokaryotic cells, such as bacterial cells, or eukaryotic cells, including mammalian (including human) cell lines such as stem cells, muscle cells, neural cells, or other kinds of mammalian cells As the field can be applied pinpoint to particular cells (such as if a cell is isolated for example) or group of cells, cell proliferation can be stimulated at a particular section of the biological material being generated, allowing to achieve the cell group with a structure necessary for a particular purpose (such as layering of a particular tissue being engineered). In a similar way, the application of the field can cause certain apoptosis in cells by causing stress in the cells, providing a further tool for shaping the biological material being created by destroying cells in certain spots of the material. Thus, by controlling the areas in which cell growth and division happens and in which cell death happens, the device 11 can cause the growth of the biological material being created in a desired direction.

The device 11 can further shape the biological material (such as human or animal tissue being created in vitro) by causing migration of the cells upon which the field is applied. Cell migration takes place in vivo during multiple processes, such as development of an embryo, immune response, cancer metastasis, and wound healing. For instance, during embryonic development, dividing cells move to specific sites to form tissues and organs. Likewise, during wound healing, macrophages and neutrophils move to the wound to kill pathogenic microorganisms, and fibroblasts move to the wound to remodel damaged tissues. Multiple cellular movement mechanism exist, such as cytoskeleton-based cell crawling and flagella-based movement, and while such movement is often driven by a chemical gradient, the movement can similarly be initiated and controlled via the application of the field by the device 11 in a monitoring-based feedback loop, providing additional tools in achieving desired tissue generation. As further described below, the progression of the cells towards desired position can be tracked using sensors such as optical sensors (e.g. a camera), and the field can be adjusted to account for the position.

In a still further embodiment, the field can be applied to achieve tissue generation in a living organism. For example, the device 11 could include an opening where a human user could insert a limb with a wound, and the field would be applied to the wound site to promote wound healing in the monitoring-based feedback loop.

While the devices 11, 51, 57 are described as being able to apply the field and monitor the effect of the field using sensors to different kinds of cellular organisms, in a further embodiment, a single device 11, 51, 57 could be used to control cellular functions and motility in multiple kinds of organisms. Thus, in one embodiment, a single device, with use of proper containers (or other kinds of enclosures), could be used to apply the field to terrestrial plants, aquatic plants, fungus, algae, eukaryotic and prokaryotic cellular cultures as well as parts of living organisms.

The device 11, 51, 57 communicates with a feedback server 14, 16 via an internetwork 12, such as the Internet or a cellular network, to obtain and adjust parameters of the field based on the obtained characteristics. In one embodiment, the feedback server 14 can be a cloud-based server. Alternatively, the server 16 can be locally or remotely located with respect to the field application device 11, 51, 57. The feedback server 14, 16 can include an identifier 18, 20 and an adjuster 19, 21. The identifier 18, 20 can utilize measurements for characteristics of the cells obtained from the field application device 11, 51, 57 to determine an identity or classification of the cells (either individually or as a collective forming a part of an organism such as a plant) based on known composition values 22, 24 of objects stored in a database 15, 17 associated with the server 14, 16. Machine learning can also be used in lieu of or in addition to a look up table of compositions and identities or classifications.

The adjuster 19, 21 utilizes data obtained from the field application device 11, 51, 57 regarding the cells and the field to determine an initial set of parameters for the field to be applied and whether the field should be adjusted to ensure an appropriate effect is reached. The adjustment can be determined using characteristic values 23, 25 of the object and field stored by the databases 15, 17 to determine new parameters for the field. In a further embodiment, ranges of cell conditions and field characteristics can be stored on the device 11, 51, 57 for use in adjusting the fields applied to the cells. Alternatively, machine learning can also be used to determine and adjust field parameters in lieu of a stored look up table of characteristic values and parameters. If different kinds of fields are applied by the same device at the same time, such as if different field generators are used to apply different fields to roots and leaves or a plant, or to cells in different portions of a tissue being generated, the initial sets of parameters and the adjustments would be determined for all the fields at the same time.

In an embodiment where a single device 11, 51, 57 can achieve multiple kinds of effects, the adjuster 19, 21 can further utilize the function 63, 64 selected by the by the user for generation of the parameters. Such functions 63, 64 can include the kind of effect that is desired, such as growth of an organism or a part of an organism or the death of an organism or the part of the organism, and the degree of the effect of the organism, such as how much growth is necessary. Similarly, when the cells are distributed over a significant volume, such as in the case of a plant or a tissue being generated, the function 63, 64 can specify specifically which cells in an organism or a group of cells need to be manipulated using the field by selecting which field generator included in an array within the device 11, 51, 57 is used to be used to apply the field, as further described with in commonly-assigned U.S. patent application, entitled “System and Method for Metamaterial Array-Based Field-Shaping,” Ser. No. ______, filed Jul. 28, 2022, pending, the entire disclosure of which is incorporated by reference. The function 63, 64 can be entered by the user through the user interface of the device 11, 51, 57 and then wirelessly provided to the adjuster 19, 21, or can be provided to the adjuster 11, 51, 57 through a further computing device, such as a mobile phone or a personal computer interfaced to the adjuster 19, 21 through the Internetwork 12.

In a further embodiment, identification or classification of the cells as well as the determination of the parameters of the field can occur on the field application devices 11, 51, 57, such as via a processor, which is described in detail below with respect to FIG. 3.

The ability to automatically determine a composition of cells and their location, including when the cells are part of a multicellular organism such as a plant, and determine and adjust parameters for field application helps to maintain progression towards the desired effects. FIG. 2 is a flow diagram showing a method 30 for controlling cell functioning and motility with the aid of a digital computer in accordance with one embodiment. The method 30 can be implemented using the system 10 of FIG. 1. A cell or a group of cells, including groups of cells that are part of a multicellular organism or of a biological material being generated in vitro, are placed within a field application device 11, 51, 57. Optionally, either before or after the cells are placed into the device 11, 51, 57, if the device 11, 51, 57 can accomplish perform multiple kinds of functions that accomplish different effects on the cells, a selection of the function 63, 64 is received (step 31), either into the device 11, 51, 57 in which the cells are positioned, or into the adjuster 19, 21 via the further communication device. The received function 63, 64 can include the desired effect on the organism, such as extraction of substances from the medium, death, or movement in a particular direction that needs to be accomplished, as well as, if multiple cells are present in the device 11, 51, 57, which of the cells need to be affected, such as which parts of a plant or which cells in a biological material need to experience the effect.

A composition or particular characteristics of at least some the cells or structures formed by those cells are be identified (step 32) via sensors. For example, one or more sensors can send signals towards the cells and information about the cells (and possibly the organism the cells are part of) is obtained via the signal, which is returned back to the sensor, and a combination of these sensors. Passive and active sensors can be used, including imaging and reflective sensors, as well as electrocurrent sensors, biosensors, volatile gas sensors, optical sensors (including a camera), chemical sensors, electrochemical sensors, acoustic sensors, and hyperspectral imaging. Likewise, if cell sampling is necessary for a sensor to be used, such as for labeling cells with fluorescent markers when a sensor employs microfluidics (such as flow cytometry, though other microfluidics techniques are also possible) to determine characteristics of the cells, the sensor would be supplemented by automated manipulators (not shown) included in the device 11, 51, 57 necessary for the sampling, the labeling, and presentation of the labeled cells for analysis by the sensors. If the sampling is used, the characteristics of the sampled cells are assigned to cells proximate to where the sampled cells were prior to their removal for analysis by the sensors. Further, three-dimensional microscopy can be used to obtain structural features of a cell at different depths of that cell. By utilizing light of different wavelengths (such as terahertz radiation, light in the visible spectrum, infrared light, ultraviolet light, though other wavelengths are also possible), one or more of the sensors performing the microscopy can obtain a full volumetric model and assessment of a cell, obtaining information about features of the cells such as both external and internal structure of the cell (such as location and size of particular organelles).

In multiple cells are placed into the device 11, 51, 57, the characteristics of all or only some of the cells can be determined. The determination of which cells are selected for determination of the characteristics can be based on the selected function 63, 64, though other ways for the device 11, 51, 57 to make the selection are possible. For example, if the selected function 63, 64, includes promotion of plant growth, the device 11, 51, 57, could determine characteristics of cells in at least a portion of leaves 59 of the plant 58 and roots 60 of the plant 58. Likewise, if the function 63, 64 function includes shaping a tissue being generated in vitro to grow in a particular direction, characteristics of the cells in the region of the tissue furthest in that direction could be determined.

Further, the characteristics of individual cells could be combined to obtain characteristics of the structures that the cells form. For example, data regarding chlorophyll content in individual cells could be combined to obtain chlorophyll distribution in a leaf 59 of a plant 58. The combining can be done by the sensor, or during subsequent processing obtained by the sensor. For example, a sensor may be able to give a value for chlorophyll content within a leaf without values for chlorophyll content within individual cells making up that leaf. Alternatively, the sensor could give content for one or more individual cells throughout the leaf, and the chlorophyll distribution for individual cells could be aggregated or extrapolated from the chlorophyll content values from individual cells.

Measures for characteristics, such as water content, fat content, density, size, and shape, pigment (such as chlorophyll) content and distribution, presence or absence as well as amount of certain markers on the surface of the cells, positions of the cells, cell wall roughness, other cell structural features (including both internal and external structure) as well as other characteristics, can be obtained via the sensors. For example, positions of a cell or a group of cells can be obtained via a camera or another optical sensor, and monitored as the cells migrate under the influence of the applied field until desired positions are reached. Likewise, chlorophyll distribution can be determined within a leaf or hyperspectral imaging can be used to determine a surface roughness or chemical composition of the leaf, which can be indicative of the health of the plant. Similarly, the sensors can be used to analyze biomarkers associated with the cells, such as cell surface markers present on the surface of the cells (though other kinds of biomarkers are also possible). Cell surface markers include molecules on the surface of the cells such as proteins expressed on the surface of the cells and carbohydrates attached to cell membrane. Cell surface markers can differ between different kinds of cells even if they have a similar function—for example, cell surface markers can be used to differentiate between hematopoietic stem cells, neural stem cells, and epidermal stem cells, as well as between differentiated cells such as adipocytes, hepatocytes, or neurons. Similarly, cell surface markers can be used to distinguish between different stages of development of the same kinds of cells. For example, exposure of phosphatidylserine (a phospholipid) on the outer leaflet of the cell membrane is a cell surface marker of apoptosis (programmed cell death). Similarly, cells that have become cancerous can display cell surface markers (such as CD24) not present when the same type of cell is not cancerous. Likewise, cell surface markers (such as CD25) of an activated T-Cell differ from the cell surface markers of an activated T-cell. Electrochemical cytosensors and sensors that are part of a flow cytometry instrument can be used to detect the cell surface markers, though other kinds of sensors are also possible.

In one embodiment, the identified characteristics can be used to classify the cells or the organism the cells are a part of. A classification can group the object into a category of organisms, such as plants, animals, protists, algae, fungus, bacteria, or even broader categories, such as eukaryote or prokaryote. The characteristics can also be used to specify a particular species of the organism, such as a particular species of plants that has a specific pigment composition. When cells that are not part of a living organisms are examined, such as cells used in tissue engineering, different chemical markers present on cell surface could be used to determine one or more of the identity of the cells and lifecycle stage they in.

Likewise, classification or identification of an object can occur via a camera or lensless digital holography, using a look up table, be provided by a user, or determined via machine learning. When used, a camera can obtain an image of the object that can be compared with a database of images to determine an identity of the object. The look up table can include characteristics, values for the characteristics, and identities or categories for the object based on the identified characteristics and values. Likewise, when a digital holography-based sensor is used, microscopic light distribution pattern produced when laser light is shined at a cell can be used to create a three-dimensional image of that cell, which can be used to determine cell's identity, such as by using machine learning or by using a lookup table of known images.

If user provided, the user can provide the characteristics of the cells or an identity of the cells or organism that the cells are a part of by entering the characteristics or identity into the field application device 11, 51, 57 or through a further computing device into the adjuster 19, 21. Alternatively, during machine learning, values for the characteristics are input to classify the cells or the organism the cells are a part of as having a particular identity or belonging to a particular category.

Initial parameters for a field applied during controlling cell function and motion can be determined (step 33) based on at least one of the characteristics of the cells (or the structures or organisms that they form), or the identity or classification of the cells (or organisms the cells are a part of), if known. Specifically, when an identity of the cells, structure, or the organism is not known, one or more of the characteristics can be used to determine a type of field and initial parameters for the determined field. The field can include a magnetic field, electric field, an electromagnetic field, or a combination of fields. Other types of fields are possible.

Meanwhile, the field parameters can include amplitude, frequency, phase, waveform, and duration, as well as other types of parameters. Values for the parameters can be determined using a look up table, which can provide field parameter values for cells or organism based on a characteristic or a combination of characteristics, or based on an identity or classification of the cells or the organism. In a further embodiment, machine learning can be used to determine the initial field parameters. The learning can be performed based on data sets of the characteristic values and parameters for fields to be applied to each of the different objects. Once the parameters are determined, the field is then applied (step 34) to the cells or organism or particular parts of the organism (as specified by the function 63, 64), based on the values of the parameters.

To maintain desired progression towards desired effect on the cells, a monitoring-based feedback loop (steps 35-39) is run (step 35). While undergoing application of the field, the cells can be monitored (step 36) continuously or at predetermined time periods to determine characteristics of the cells (including condition of the organism or structure the cells are a part of). For example, characteristics of the cells can be monitored, including temperature, positon, impedance, hyperspectral imaging characteristics, acoustic sensing characteristics, visible and infrared imaging characteristics, as well as biomarkers such as cell surface markers detectable through flow cytometry instrumentation or other sensors. Thus, for example, if the field is being applied to a terrestrial plant, the characteristics monitored can include the degree of growth the plant has experienced and the pigment composition in parts of the plant. Likewise, if individual cells or group of cells are being monitored as part of tissue generation, how far the cells have moved in a desired direction and whether the cells have started exhibiting a desired cell function, such as division, growth, extraction of substances from the medium 62, differentiation into a desired cell type, or apoptosis. Parameters of the applied field can also be monitored (step 36), including wavelength, frequency, phase, amplitude, waveforms, and duration. If at any time, field application becomes unnecessary (step 37), such as if the desired goals have been detected achieved based on the monitored characteristics, if the cells are detected to have been removed from the device, or if a user command to stop application of the field, monitoring of the field ends, stopping (step 38) the feedback loop and stopping the method 30.

However, if the application of the field does not become unnecessary, the monitored characteristics of the cells and parameters of field can be used to determine whether the field needs to be adjusted (step 37). If the progression towards the desired goal is satisfactory (which can be determined based on predetermined benchmarks to which the characteristics of the cells or parameters of the field are compared), no adjustment may be necessary (step 39). For example, if a plant is growing at an expected rate or the cells are moving at a desired direction at an appropriate speed, no change to the field is made.

If the progression towards the desired goal is not satisfactory, the field is adjusted (step 39). For example, if a plant growth is too slow, or if instead of growth cells exhibit apoptosis, the field is adjusted. The field changes can be made manually or automatically. In one embodiment different formulas can be used to determine new parameter values based on the monitored characteristics of the cells, as well as a graph of cell characteristics and calibration of the fields. The chart can include values for the listed characteristics with standard deviations and known progression of time for each kind of organism or cell with a particular characteristic or combination of characteristics to achieve the desired result. In a different embodiment, machine learning can be used to determine new values for the field parameters.

After the parameters are changed, the field is applied (step 34) to the object using the adjusted parameters and the feedback process continues (step 34). For example, a magnetic field can be changed by moving the magnets closer to or away from the object, or moving the magnets relative to one another. Movement of the magnets can be manual or automated.

As multiple kinds of fields can be applied to the same organism or a group of cells at the same time, multiple feedback loops (steps 35-39) could be running at the same time. For example, in the case of a plant where a magnetic field is being applied to the roots of the plant and an electric field is being applied to leaves of the plants, a single feedback loop (steps 35-39) could be running for cells in the leaves and for cells in the roots. Further, the field applied to one group of cells could be adjusted based on characteristics of another group of cells. For example, even if a field is only applied to roots of the plants, a change in health of the leaves of that plant could require adjustment of the field being applied to the roots.

The device 11, 51, 57 can vary in size depending on the size of cellular organisms whose function, motility, or both is manipulated. FIG. 3 is a block diagram showing a top view of a device 11, 51, 57 for feedback-based field application for use in the system 10 and method 30 for controlling cell functioning and motility in accordance with one embodiment. The device 11, 51, 57 can include a receptacle 40 in which the cells being manipulated are placed. The receptacle 40 can include a container, pan, or other type of receptacle for holding the cell and the medium 52, 61, 62 in which they are located. In a further embodiment, instead of receiving the medium 52, 61, 62 and the cells directly, the receptacle 40 can receive the scaffolding 52, 61, 62 in which the cells and the medium 52, 61, 62 are positioned. In one embodiment, the receptacle 40 is placed into a standalone housing (not shown), similar to a microwave, to initiate the field application, or alternatively, can be incorporated into another structure, such as a incubator or a fixture in a greenhouse. In a further embodiment, the receptacle 40 does not have to surround the cells from all directions; for example, if the cells are in a terrestrial plant 58 growing in the ground, the receptacle 40 can be placed around the plant 58 similar to a fence. Likewise, if the cells that need to be manipulated are part of a living organism that either does not fit within or should not be placed entirely within the enclosure, such as in a case of a human arm that has a wound, the receptacle 40 can include an opening into which the part of the organism that does get manipulated is inserted.

One or more field generators 42 a,b, 43 a,b can be positioned with respect to the receptacle 40. The field generators can each include one or more magnets, one or more electrodes, or both magnets and electrodes. For example, electrodes 43 a,b can be positioned on a bottom side of the receptacle, along an interior surface, to generate a pulsed electric field. Other positions of the electrodes are possible, including on opposite sides (not shown) of the receptacle 40. When placed in a position other than the bottom of the receptacle, the electrodes can be affixed to walls of the standalone housing or walls of a housing, such as an appliance. However, at a minimum, the electrodes should be touching or at least be proximate to the cells.

The field application device 11, 51, 57 can also include at least one magnet 42 a, b, such as an electromagnet, a permanent magnet, or a combination of magnets, to generate an oscillating magnetic, electric or electromagnetic field. Time-varying magnetic fields can be used to create electric fields and vice-versa. The magnets can be positioned along one or more sides of the receptacle 40, or can be affixed to the receptacle 40 or the housing in which the receptacle is placed. In a further embodiment, the magnets can be remotely located from the receptacle 40 and the field emitted from the magnets can be applied to the cells via one or more transducers.

Further, at least one closed-loop monitoring sensor 41 can be provided adjacent to the receptacle 40 on one or more sides. Alternatively or in addition, a sensor can be affixed to the housing, on an interior surface, in which the receptacle is placed for use. The monitoring sensors 41 can be active and passive, and can include reflective sensors, electric sensors, acoustic sensor, optical sensors, electrochemical sensors, thermal sensors and imagers, hyperspectral sensors, biosensors, volatile gas sensors, and sensors that are part of flow cytometry instrumentation. If sampling of the cells is necessary for analysis and by any of the sensors 41 and the sampled cells must be removed from other cells, the device 11, 51, 57 can further include manipulators (not shown) necessary to perform the automated sampling and other pre-processing necessary for preparation of the cells for analysis by the sensors. Still other types of sensors are possible.

Different field can be applied to different parts of the same organism, group of cells, or biological material such as a tissue being generated. For example, magnetic fields have a greater effect on the roots 60 of the plants while electric fields have a greater effect on the upper part of the plants 58, such as leaves 59, and thus, in a device 11, 51, 57 that manipulates function of cells in terrestrial plants 58, electrodes could be positioned 43 a,b in a portion of the device 11, 51, 57 proximate to where the leaves 59 would be located and magnets 42a,b could be located proximate to where the roots 60 are located. Likewise, different sensors 41 could be positioned relative to where the cells they are intended to analyze would be located when the cells are inside the device 11, 51, 57. The location of the field generators 42a,b, 43a,b, and the sensors 41 could be fixed within the device 11, 51, 57, or could be adjustable, either automatically, with machinery inside the device 11, 51, 57 that is being controlled based on user commands, or by the user manually.

An electrical control unit 45 can be a processor that is interfaced to the sensors 41, magnets 42 a,b, and electrodes 43 a,b to communicate during the feedback process. Specifically, the processor can determine an identity of or classify cells based on measurements from the sensors 41, as well as identify parameters for the field to be applied based on the identity or classification. The processor can also instruct the sensors 41 to measure characteristics of the cells whose function or motility (or both function and motility) are being manipulated, and in turn, receive the measured values as feedback for determining if new parameters of the field are needed and if so, values of the parameters. Based on the feedback from the sensors, the processor can communicate the new parameter values with the magnets and electrodes to change the field applied to the cells for maintaining progress towards the desired goals.

In a further embodiment, the processor 45 can obtain data from the sensors, electrodes, and magnets for providing, via a wireless transceiver included in the device 11, 51, 57, to a cloud-based or dedicated server 14, 16 for determining an identity or classification of the object, determining initial parameters for the field, and identifying new field parameters for adjusting the field. When performed in on the server 14, 16, the data set of cell identities and classifications, initial parameters, and guidelines for adjusted parameters can be utilized by different users. In contrast, when the processor of the device performs such actions, the data sets are specific to that field application device 11, 51, 57.

While the description above focuses on controlling cell function and motility, the system and process described above can also be applied to different kinds of objects, including other objects, including, raw, preserved or cooked foods, blood, embryos, vaccines, probiotics, medicines, sperm, tissue samples, plant cultivars, cut flowers and other plant materials, biological samples of plants, non-biologicals, such as hydrogel materials, material that can be impacted by water absorption, such as textiles, nylons and plastic lenses and optics, fine instruments and mechanical components, heat exchangers, and fuel, as well as ice as described in commonly-assigned U.S. patent application, entitled “System and Method for Feedback-Based Beverage Supercooling,” Serial No. ______, filed Jul. 28, 2022, pending; ice as described in commonly-assigned U.S. patent application, entitled “System and Method for Controlling Crystallized Forms of Water,” Ser. No. ______, filed Jul. 28, 2022, pending; organic items as described in commonly-assigned U.S. patent application, entitled “System and Method for Feedback-Based Nucleation Control,” Ser. No. ______, filed Jul. 28, 2022, pending and commonly-assigned U.S. patent application, entitled “Feedback-Based Device for Nucleation Control,” Ser. No. ______, filed Jul. 28, 2022, pending; colloids as described in commonly-assigned U.S. patent application, entitled “System and Method for Feedback-Based Colloid Phase Change Control,” Ser. No. ______, filed Jul. 28, 2022, pending; lab grown material, including meat, as described in commonly-assigned U.S. patent application, entitled “System and Method for Controlling Cellular Adhesion with the Aid of a Digital Computer,” Ser. No. ______, filed Jul. 28, 2022, pending; and food as described in commonly-assigned U.S. patent application, entitled “System and Method for Metamaterial Array-Based Field-Shaping,” Ser. No. ______, filed Jul. 28, 2022, pending, the disclosures of which are incorporated by reference. Further, a receptacle packaging described in commonly-assigned U.S. patent application, entitled “An Electrode Interfacing Conductive Receptacle,” Ser. No. ______, filed Jul. 28, 2022, pending, the disclosure of which is incorporated by reference, can be used to hold the cells to which the field is being applied to prevent the cells from touching electrode contacts.

While the invention has been particularly shown and described as referenced to the embodiments thereof, those skilled in the art will understand that the foregoing and other changes in form and detail may be made therein without departing from the spirit and scope of the invention.

What is claimed is:

1. A feedback-based method for controlling cell functioning and motility, comprising:
using by a controller one or more sensors to determine characteristics of one or more of the cells within a receptacle at multiple time points;
determining by the controller parameters for one or more fields to be applied to one or more of the cells at the multiple time points via one or more field generators positioned with respect to the receptacle based on the characteristics determined at those time points, each field generator comprising one or more of a magnet, transducer, electromagnet, or a pair of electrodes, the parameters comprising at least one of amplitude, frequency, wavelength, phase, waveform, and duration; and
controlling by the controller application of the one or more fields by the one or more field generators based on the parameters, wherein each of the applied fields causes a change in at least one of functioning and motility of the cells to which that field is applied.

2. A method for controlling cell functioning and motility according to claim 1, wherein the one or more sensors comprise one or more of electromagnetic sensors, hyperspectral imaging sensors, impedance sensors, chemical sensors, biosensors, optical sensors, acoustic sensors, electrochemical sensors, microfluidics sensors, and volatile gas sensors.

3. A system for controlling cell functioning and motility according to claim 1, wherein the plurality of cells are comprised in a plant and the cells to which the field is applied are comprised in at least one or more leaves of the plant, one or more portions of a stem of the plant, and one or more of the roots of the plant.

4. A method for controlling cell functioning and motility according to claim 3, wherein at least some of the sensors measure pigment composition of the leaves.

5. A method for controlling cell functioning and motility according to claim 3, wherein the application of one or more of the fields causes the roots to accelerate extraction of one or more substances from a medium in which the roots are positioned.

6. A method for controlling cell functioning and motility according to claim 5, wherein the medium is one of a soil and a hydroponic medium.

7. A method for controlling cell functioning and motility according to claim 1, wherein the application of one or more of the fields causes one or more of the cells to accelerate extraction and increase retention of one or more substances from a medium in which the cells are positioned.

8. A method for controlling cell functioning and motility according to claim 1, wherein the extracted substances are metals.

9. A method for controlling cell functioning and motility according to claim 1, wherein the application of one or more of the fields causes at least one of a growth of the cells to which that field is applied, a change in proliferation of the cells to which that field is applied, and death of the cells to which that field is applied.

10. A method for controlling cell functioning and motility according to claim 1, wherein the application of the one or more fields causes at least some of the cells to at least one of move or grow into a direction.

11. A method for controlling cell functioning and motility according to claim 10, wherein the direction is controlled by the parameters of the one or more fields.

12. A method for controlling cell functioning and motility according to claim 1, wherein the cells are comprised in a seed and the application of one or more of the fields causes the seed to germinate.

13. A method for controlling cell functioning and motility according to claim 1, wherein the cells are selected from the group consisting one or more of plant cells, eukaryotic cells, prokaryotic cells, mammalian cells, algae cells, fungal cells, and protist cells.

14. A method for controlling cell functioning and motility according to claim 1, wherein at least some of the sensors perform three-dimensional microscopy and at least one of the characteristics of one of the cells comprises structural features of that cells.

15. A method for controlling cell functioning and motility according to claim 14, wherein a depth of the structural features depends on a wavelength of a light used during the microscopy.

16. A method for controlling cell functioning and motility according to claim 15, wherein the light comprises one or more of terahertz radiation, visible spectrum light, infrared light, and ultraviolet light.

17. A method for controlling cell functioning and motility according to claim 14, wherein the structural features comprise one or more of a location and size of one or more organelles within that cell.

18. A method for controlling cell functioning and motility according to claim 1, wherein the characteristics comprise one or more of the cells producing one or more biomarkers.

19. A method for controlling cell functioning and motility according to claim 1, wherein the characteristics comprise one or more of the cells producing one or more biomarkers.

20. A method for controlling cell functioning and motility according to claim 1, wherein the one or more cells comprise a plurality of groups of cells and the field applied to one of the cell groups differs from the field applied to another group of the cells.
